(19) United States
(12) Patent Application Publication
(10) Pub. No.: US 2016/0206570 A1
(43) Pub. Date: Jul. 21, 2016

(54) CARDIOPROTECTANT TO REDUCE DAMAGE FROM HEART ATTACK

(71) Applicants: ARIZIBA BOARD OF REGENTS ON BEHALF OF THE UNIVERSITY IF ARIZONA, Tuscon, AZ (US); NUVOX PHARMA, Tucson, AZ (US)

(72) Inventors: Evan C. Unger, Tucson, AZ (US); Douglas F. Larson, Tucson, AZ (US)

(21) Appl. No.: 14/913,987

(22) PCT Filed: Sep. 3, 2014
(86) PCT No.: PCT/US2014/053922

§ 371 Date: Feb. 23, 2016

Related U.S. Application Data
(60) Provisional application No. 61/873,276, filed on Sep. 3, 2013.

(51) Int. Cl. A61K 31/02; A61K 47/24; A61K 47/44

(52) U.S. Cl. CPC A61K 31/02; A61K 47/44; A61K 47/24

(57) ABSTRACT

A composition for treating a subject who has incurred or is incurring damage heart, where the composition comprises a perfluorocarbon and a lipid.

### FIELD OF THE INVENTION

The invention is directed to a composition for administration in a subject before or following a heart attack

### BACKGROUND OF THE INVENTION

There are presently no approved cardioprotectants.

### SUMMARY OF THE INVENTION

A composition is disclosed for treating a subject who has incurred or is incurring damage to the heart, where that composition comprises a perfluorocarbon in combination with a lipid. In certain embodiments, the perfluorocarbon consists essentially of dodecafluoropentane, where other perfluorocarbons are present at a level less than about 0.10 weight percent.

A method is disclosed for treating a subject who has incurred or is incurring damage to the heart, where the method includes administering to the subject a therapeutically effective amount of Applicants' composition.

### BRIEF DESCRIPTION OF THE DRAWINGS

The invention will be better understood from a reading of the following detailed description taken in conjunction with the drawings in which like reference designators are used to designate like elements, and in which:

FIG. 1 graphically illustrates a decrease in left ventricular myocardial damage in mice when treated with a Dodecafluoropentane emulsion prior to coronary artery occlusion.

### DETAILED DESCRIPTION OF PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Presently, there are no approved cardioprotectants. It is thought that microbubbles can carry more oxygen than liquids. Among many perfluoro compounds, Dodecafluoropentane emulsion (DDFPe) is a unique material with quasi-bubble properties. DDFPe carries far more oxygen per unit volume than liquid fluorocarbons. The boiling point of DDFPe is about 29° C. The emulsion particles are about 250 nm in diameter. After IV administration, DDFPe circulates throughout the body and during passage through the pulmonary capillary bed, imbibes oxygen, and releases the oxygen in tissues with low pO₂.

For treatment of heart attack DDFPe is administered IV and circulates throughout the body, including the myocardial circulation. In certain embodiments, the DDFPe can be administered IV as a bolus, a slow IV push, or as a sustained infusion.

In the at risk myocardium where there is still some degree of collateral blood flow, DDFPe delivers oxygen to keep myocardial tissue alive. In an occlusive model of myocardial infarction in mice (ligation of LAD) a single administration of DDFPe (0.6 cc/kg) shortly after the time of occlusion decreased the amount of left ventricular myocardial damage from 30% to 12% of the heart.

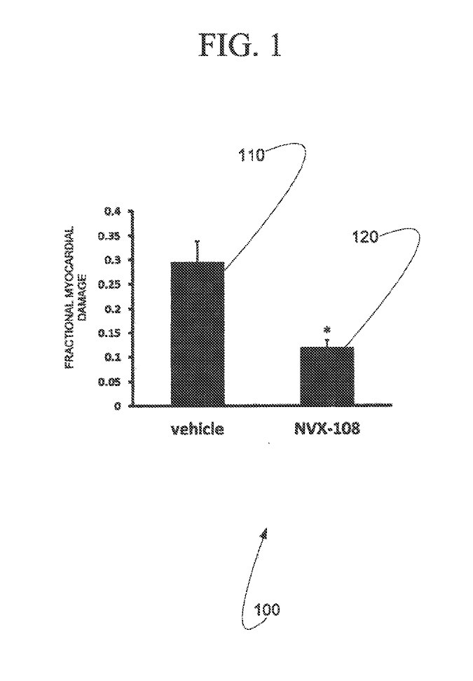

Referring now to FIG. 1, graph 100 graphically shows the effect of DDFPe on left ventricular myocardial damage in mice. Left anterior descending coronary artery was ligated. There were 5 mice in control group (vehicle) and 5 mice treated with DDFPe (NVX-108). Single dose of 0.6 cc per kg (2% w/vol DDFP) was administered IV. Animals were euthanized 24 hours following coronary artery occlusion and left ventricular myocardial damage quantitated post mortem. Curve 110 shows results from control animals having about 30% myocardial damage. Curve 120 shows results from animals treated with NVX-108, and having about 12% myocardial damage (p<0.01).

The prevention of chemical breakdown is important for the long-term physical stability of the NVX-108 (DDFPe) formulation for use as a cardioprotectant. A buffer is provided that stabilizes the viscosity of the suspending medium surrounding an emulsion of a fluorocarbon material. The addition of a 0.01 M phosphate buffer to NVX-108, a dodecafluoropentane emulsion (DDFPe), stabilizes the pH.

Applicants further discovered that this buffer actually functions to maintain the desired viscosity of the NVX-108 emulsion. Furthermore, the buffer prevents an increase in the osmotic concentration of the formulation over time. Due to its ability to organize in aqueous solution and form a quasi lattice-work to support the emulsion droplets, sucrose (30% w/v) is employed as the viscosity enhancer in this formulation.

When a sucrose molecule hydrolyzes, it becomes a molecule of fructose and a molecule of glucose; thus, potentially doubling the overall solute concentration of the aqueous phase. In addition, fructose and glucose destabilize the sucrose scaffolding which in turn decreases the viscosity of NVX-108. Maintaining the integrity of the initial sucrose “structure” positively contributes to the physical stability of the formulation by maintaining a constant osmotic concentration, and the inherent molecular lattice that is specific to sucrose in water, to provide a 2-fold increase in viscosity.

As a general matter, the present invention encompasses a method for improving cardiac tissue oxygenation in a subject at risk for ischemic tissue damage. The method comprises administering an effective amount of a composition comprising a perfluorocarbon emulsion to the subject prior to an event, where that event be a medical procedure, surgical procedure, or a trauma event, that results in the subject being at high risk of ischemic damage of cardiac tissues.

Applicants' composition can be used to reduce cardiac tissue dame where that damage arises from ischaemia, ischaemia/reperfusion injury, hypoxia, increased cardiac workload or cardiac stress, increased pressure on the heart, a cardiotoxic substance, infection, or a maladaptive response of the heart to injury or disease.

The present invention describes methods and combinations that may be used to reduce tissue damage resulting from an ischemic event in a subject. The methods comprise administering a composition or a combination comprising an oxygen transport substance to the subject. In a preferred embodiment, the oxygen transport substance is a composition comprising a perfluorocarbon emulsion. In addition, the methods and combinations are effective in reducing infarct volume. Methods of the invention also encompass reducing tissue damage from cardiac infarct. Advantageously, the methods and combinations are effective for pretreating subjects at high risk of an ischemic event.

In one embodiment, the invention encompasses a method for reducing the infarct volume in a tissue of a subject undergoing ischemia resulting from an ischemic event. The method comprises administering an effective amount of a composition comprising an oxygen transport substance to the subject, wherein the infarct volume is reduced without resolving the ischemic event. In a preferred embodiment, the oxygen transport substance is a composition comprising a perfluorocarbon emulsion.

Generally speaking, the oxygen transport substance may be administered to the subject before the ischemic event is resolved. Stated another way, the oxygen transport substance may be administered to reduce infarct volume even though normal blood flow, blood pressure, or oxygenation levels in the tissue have not been restored.

As used herein, the term “ischemic′” may refer to a restriction in blood supply, generally due to factors in the blood vessels, with resultant damage or dysfunction of tissue due to inadequate oxygenation. Ischemia may be caused by an “ischemic event.”

In some embodiments, infarct volume may be decreased about 100%, 90%, 80%, 70%, 60%, 50%, 40%, 30%, 20%, or 10% compared to an infarct volume when no oxygen transport substance is administered during a comparable ischemic event. For instance, infarct volume may be decreased by about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 6, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10% compared to an infarct volume when no oxygen transport substance is administered during a comparable ischemic event. In an exemplary embodiment, infarct volume may be decreased by about 70 to about 90% compared to an infarct volume when no oxygen transport substance is administered during a comparable ischemic event.

In particular embodiments, infarct volume may be decreased to about 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0 or about 3.1% of the tissue when an oxygen transport substance of the disclosure is administered, compared to an infarct volume of about 3.2% or greater when no oxygen transport substance is administered. In other embodiments, infarct volume may be decreased to about 0, 0.1, 0.2, 0.3, 0.4, 0.5 or about 0.6% of the tissue when an oxygen transport substance of the disclosure is administered, compared to an infarct volume of about 3.2% or greater when no oxygen transport substance is administered. In yet other embodiments, infarct volume may be decreased to about 0.5, 0.6, 0.7, 0.8, 0.9, 1, or about 1.1% of the tissue when an oxygen transport substance of the disclosure is administered, compared to an infarct volume of about 3.2% or greater when no oxygen transport substance is administered. In additional embodiments, infarct volume may be decreased to about 1, 1.1, 1.2, 1.3, 1.4, 1.5, or about 1.6% of the tissue when an oxygen transport substance of the disclosure is administered, compared to an infarct volume of about 3.2% or greater when no oxygen transport substance is administered. In other embodiments, infarct volume may be decreased to about 1.5, 1.6, 1.7, 1.8, 1.9, 2, or about 2.1% of the tissue when an oxygen transport substance of the disclosure is administered, compared to an infarct volume of about 3.2% or greater when no oxygen transport substance is administered. In still other embodiments, infarct volume may be decreased to about 2, 2.1, 2.2, 2.3, 2.4, 2.5, or about 2.6% of the tissue when an oxygen transport substance of the disclosure is administered, compared to an infarct volume of about 3.2% or greater when no oxygen transport substance is administered. In additional embodiments, infarct volume may be decreased to about 2.5, 2.6, 2.7, 2.8, 2.9, 3.0 or about 3.1% of the tissue when an oxygen transport substance of the disclosure is administered, compared to an infarct volume of about 3.2% or greater when no oxygen transport substance is administered. In yet other embodiments, infarct volume may be decreased to about 0, 0.5, 1, 1.5, 2, 2.5, 3.0 or about 3.1% of the tissue when an oxygen transport substance of the disclosure is administered, compared to an infarct volume of about 3.2% or greater when no oxygen transport substance is administered.

The methods of the present disclosure comprise administering an oxygen transport substance to a subject. Non-limiting examples of a subject in need of an oxygen transport substance may be a rodent, a human, a livestock animal, a companion animal, a laboratory animal, or a zoological animal.

In one embodiment, the subject in need of an oxygen transport substance may be a lab animal. Non-limiting examples of a lab animal include a rabbit, a mouse, a guinea pig, a hamster, or a rat. In another embodiment, the subject in need of an oxygen transport substance may be a rodent, e.g. a mouse, a rat, a guinea pig, etc.

In yet another embodiment, the subject in need of an oxygen transport substance may be a livestock animal. Non-limiting examples of suitable livestock animals may include pigs, cows, horses, goats, sheep, llamas and alpacas.

In another embodiment, the subject in need of an oxygen transport substance may be a companion animal. Non-limiting examples of companion animals may include pets such as dogs, cats, rabbits, and birds.

In still yet another embodiment, the subject in need of an oxygen transport substance may be a zoological animal. As used herein, a “zoological animal” refers to an animal that may be found in a zoo. Such animals may include non-human primates, large cats, wolves, and bears.

In an exemplary embodiment, the subject in need of an oxygen transport substance may be a human. In certain embodiments, the human subject may be undergoing a medical procedure that increases the risk of a cardiac infarct. Non-limiting examples of medical procedures that may increase the risk of vessel occlusion may include major or minor surgical or catheter based procedure or interventional which may cause hemorrhage or the formation of blood clots leading to cardiac infarct.

In certain embodiments, the invention comprises a method for reducing vessel occlusion during a medical procedure that increases the risk for vessel occlusion, the method comprising administering an effective amount of a dodecafluoropentane emulsion to a subject before the medical procedure is performed.

In other preferred embodiments, the dodecafluoropentane emulsion is administered in a time ranging from immediately after the onset of symptoms of an occluded blood vessel to 24 hours after the onset of symptoms of an occluded blood vessel.

In yet other preferred embodiments, the dodecafluoropentane emulsion is administered to the subject intravenously.

In other preferred embodiments, a solution of about 1% to about 5% w/v of the dodecafluoropentane emulsion is administered to the subject in an amount of about 0.2 ml_ to about 1 ml_per kilogram of the subject.

In yet other preferred embodiments, a solution of about 2% w/v of the dodecafluoropentane emulsion is administered to the subject in an amount of about 0.01 ml_per kilogram to about 1 ml per kilogram of the subject. In other preferred embodiments, the dodecafluoropentane emulsion improves the oxygenation to the tissue such that the infarct volume is reduced without increasing incidence of brain hemorrhage.

In yet other preferred embodiments, the dodecafluoropentane emulsion is administered in combination with an anticoagulant.

In other embodiments, the dodecafluoropentane emulsion is administered in combination with a thrombolytic drug selected from the group consisting of tissue plasminogen activators, antistreptase, streptokinase, urokinase, and combinations thereof.

In yet other embodiments, the dodecafluoropentane emulsion is administered in combination with surgical techniques, such as and without limitation cardiac surgery.

In preferred embodiments, the oxygen transport substance comprises one or more perfluorochemicals (PFCs). PFCs may be liquid perfluorochemicals that dissolve oxygen. Non-limiting examples of liquid PFCs that dissolve oxygen and may be used as an oxygen transport substance include perfluorooctyl bromide, perfluorooctyl dibromide, bromofluorocarbons, perfluoroethers, Fluosol DA™, F-44E, 1,2-bisperfluorobutyl-ethylene, F-4-methyl octahydroquinolidizine, 9 to 12 carbon perfluoro amines, perfluorodecalin, perfluoroindane, perfluorotrimethyl bicyclo [3,3,1] nonane, perfluoromethyl adamante, and perfluorodimethyl adamantane.

PFCs may also be a gas used to deliver oxygen in the body of a subject. Particularly useful is a PFC gas that has been formulated into microbubbles. Microbubbles comprising PFCs are known in the art and are disclosed in, for example, U.S. Pat. Nos. 5,393,524, 5,409,688, 5,558,854, 5,558,855, 5,595,723, and 5,558,853, all of which are incorporated herein by reference. Non-limiting examples of PFC gases that may be formulated into microbubbles include dodecafluoropentane (DDFPe), sulfur hexafluoride, pentane, hexafluoropropylene, octafluoropropane, hexafluoroethane, octafluoro-2-butyne, hexafluorobuta-1,3-diene, isoprene, octafluorocyclobutane, decafluorobutane, cis-2-pentene, dimethyl sulfide, ethylarsine, bromochlorofluoromethane, trans-2-pentene, 2-chloropropane, hexafluorodisulfide, ethyl mercaptan, diethylether, ethylvinylether, valylene, trisfluoroarsine, furfuyl bromide, cis-propenyl chloride, bytyl fluoride, 1,1 dichloroethane, isopropyl methyl ether, isopropylamine, methylfomate, 2-acetyl-furan, ethylenefluoride, 1-pentene, isopropylacetylene, perfluoropentane, isopentane, vinyl ether, 2-butyne, 1,4-pentadiene, tetramethyl silane, dimethyl phosphine, dibromodifluoromethane, 2-chloro-propene, difluroiodomethane, acetaldehyde, trimethyl boric, 3-methyl-2-butene, 1,1 dimethylcyclopropane, aminoethane, vinyl bromide, disilanomethane, trichlorofluoromethane, bromofluoromethane, trifluorodichloroethane, perfluoropentene, and other fluorine containing hydrocarbons. In preferred embodiments, the oxygen transport substance may be microbubbles comprising the PFC dodecafluoropentane (DDFPe).

The preferred fluorocarbons useful as an oxygen therapeutic have a boiling point between about room temperature and at about or near physiological temperature. In one embodiment, the fluorocarbon has a boiling point of below about 100° C. The preferred fluorocarbon is perfluoropentane with perfluoroisopentane being particularly preferred. Other materials include n-perfluoropentane, perfluoropropane (bp −36.7° C.), perfluorobutane (bp=−1.7° C.), perfluorocyclohexane (bp 59-60° C.), perfluoromethylcyclopentane (bp 48° C.), n-perfluorohexane (bp 58-60° C.), perfluorocyclopentane (bp 45° C.) and perfluorotriethylamine (bp 68-69° C.).

Microbubbles comprising PFCs capable of transporting oxygen in the blood are smaller than red blood cells, and can flow through partially obstructed vessels to deliver large amounts of oxygen to oxygen-starved tissues or organs. Methods of formulating microbubbles comprising PFCs are known in the art, and are disclosed in, for example, U.S. Pat. Nos. 5,393,524, and 5,558,855, all of which are incorporated herein by reference. In essence, microbubbles comprising PFC gas are prepared by a phase-shift technology whereby an emulsion of liquid PFC droplets is prepared in a cool environment, and then when infused or injected into the body of an individual, the droplets become vaporized gas microbubbles comprising a PFC gas. (a) emulsion

As used herein, the term “emulsion” may refer to a colloidal dispersion of one immiscible liquid dispersed in another liquid in the form of droplets, whose diameter, in general, exceeds approximately 100 nm and which is typically optically opaque, unless the dispersed and continuous phases are refractive index matched. In general, an emulsion of the invention comprises the dispersed PFC droplets and an amphiphilic material in a continuous phase.

The continuous phase of the colloidal dispersion of the present invention may be an aqueous medium. As used herein, the term “aqueous medium” may refer to a water-containing liquid which may contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. The amphiphilic material may be a biocompatible protein, a fluorine-containing surfactant, polyoxypropylenepolyoxyethylene glycol nonionic block copolymers, and synthetic surfactants.

In some embodiments, the composition of the invention may comprise a surfactant. Non-limiting examples of surfactants that may be used in the composition of the invention may include various commercial anionic, cationic, and nonionic surfactants, including Tweens, Spans, Tritons, and the like, phospholipids, cholesterol, PLURONIC F-68®, HAMPOSYL L30 ® (W.R. Grace Co., Nashua, N.H.), sodium dodecyl sulfate, Aerosol 413 (American Cyanamid Co., Wayne, N.J.), Aerosol 200 (American Cyanamid Co.), LIPOPROTEOL LCO (Rhodia Inc., Manmmoth, N.J.), STANDAPOL SH 135 ® (Henkel Corp., Teaneck, N.J.), FIZUL 10-127 ® (Finetex Inc., Elmwood Park, N.J.), and CYCLOPOL SBFA 30 ® (Cyclo Chemicals Corp., Miami, Fla.), amphoterics, such as those sold with the trade names: DERIPHAT 170 ® (Henkel Corp.), LONZAINE JS® (Lonza, Inc.), NIRNOL C2N-SF CRS (Miranol Chemical Co., Inc., Dayton, N.J.), AMPHOTERGE W2 ® (Lonza, Inc.), and AMPHOTERGE 2WAS (Lonza, Inc.), non-ionic surfactants, such as those sold with the trade names PLURONIC F-68 ® (BASF Wyandotte, Wyandotte, Mich.), PLURONIC F-127 ® (BASF Wyandotte), BRIJ 35 ® (ICI Americas; Wilmington, Del.), TRITON X-100 CRS (Rohm and Haas Co., Philadelphia, Pa.), BRIJ 52 ® (ICI Americas), SPAN 20 ® (ICI Americas), GENEROL 122 ES® (Henkel Corp.), TRITON N42 ® (Rohm and Haas Co.), TRITON N-101 ® (Rohm and Haas Co.), TRITON X-405 ® (Rohm and Haas Co.), TWEEN 80 ® (ICI Americas), TWEEN 85 ® (ICI Americas), BRIJ 56 ® (ICI Americas) and the like, 1,2-dipalmitoyl-sn glycerol-3-phosphoethanolamine-N-4-(p-maleimidophenyl)butyramide, amine-PEG2000-phosphatidylethanolamine, PEG Telmer B, phosphatidylethanolamine, acacia, cholesterol, diethanolamine, glyceryl monostearate, lanolin alcohols, lecithin, including egg-yolk lecithin, mono- and di-glycerides, mono-ethanolamine, oleic acid, oleyl alcohol, poloxamer, peanut oil, palmitic acid, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. The above surfactants may be used alone or in combination in the composition of the invention.

Emulsions of fluorocarbons may be prepared, in some embodiments, using fluorosurfactants such as fluorinated phospholipids. For instance, in one embodiment, the surfactant is PEG-Telomer-B. In an exemplary embodiment, the composition comprises DDFPe with PEG-Telomer-B. Phospholipids are also useful for preparing emulsions and may comprise one or more different phospholipids and also fatty acids. Chain length in phospholipids may vary from about 12 to about 20 carbon atoms in length. The alkyl groups may be saturated or unsaturated. Preferably if phospholipids are employed, two or more lipids are employed.

For example, dipalmitoylphosphatidylcholine can be mixed with dipalmitoylphosphatidylethanolamine-PEG (DPPE-PEG). In certain embodiments, the PEG-ylated lipid is usually mixed between 1 and 10 mole percent with the non-PEG-ylated lipid. PEG-ylated phospholipid 1 comprises a lipid moiety in combination with a PEG moiety. In the illustrated PEG-ylated phospholipid 1, the lipid moiety comprises dipalmitoylphosphatidylethanolamine, and the PEG moiety comprises PEG having a number average molecular weight of about 5000 Daltons.

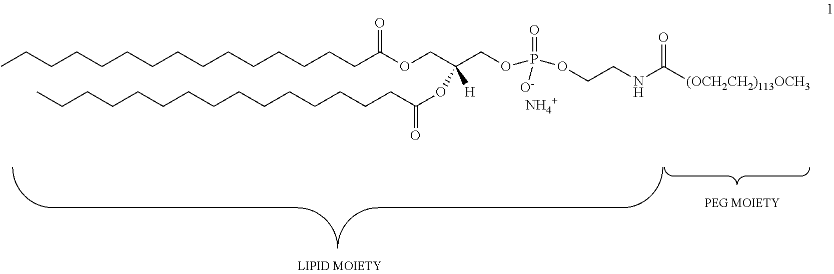

Preferably the emulsion contains two lipids, a neutral phospholipid and a second PEG-ylated phospholipid or a PEG-ylated lipid which is not a phospholipid. The PEG-ylated lipid may comprise between 1% and 100% of the total lipid in the emulsion. In certain embodiments, the PEG-ylated lipid loading is between about 1% and about 20% of the total lipid in the emulsion. In certain embodiments, the PEG-ylated lipid loading is between about 5 and about 10% of the total lipid in the emulsion.

In certain embodiments, the number average molecular weight of the PEG group affixed to the lipid is between about 100 Daltons to about 20,000 Daltons. In certain embodiments, the number average molecular weight of the PEG group affixed to the lipid is between about 1,000 Daltons to about 10,000 Daltons. In certain embodiments, the number average molecular weight of the PEG group affixed to the lipid is between about 2,000 Daltons to about 5,000 Daltons.

In certain embodiments, a non-PEG moiety portion of the lipids in the emulsion comprises from about 10 carbons to about 24 carbons in length. In certain embodiments, the lipids in the emulsion comprise from about 12 carbons to about 22 carbons in length. In certain embodiments, the lipids in the emulsion comprise from about 14 to about 20 carbons in length. Saturated and unsaturated phospholipids (and lipids other than phospholipids) may also be used in the invention and mixtures thereof.

In certain embodiments, lipids wherein the fatty acyl chains are replaced by fatty ether chains, so called ‘ether lipids’, are utilized in lieu of either the neutral phospholipid, the PEG-ylated phospholipid or both. They may also be employed as part of a mixture of phospholipids and lipids employed to stabilize the emulsion. The inventors have discovered that careful selection of the lipids may be employed to create stable emulsions of dodecafluoropentane (DDFP) and fluorocarbons and these afford effective transport of oxygen.

Cholesterol and derivatives of cholesterol such as cholesterol-acetate may be included in the emulsion. The emulsion may contain a cationic (dipalmitoylphosphatidylethylcholine) or anionic lipid (e.g. dipalmitoylphosphatidic acid) or a glycosylated lipid. The lipids or surfactants are mixed with the fluorocarbon and homogenized to prepare an emulsion. One or more viscosity modifying agents may also be included in the emulsion.

The emulsion may also comprise various additives to assist in stabilizing the dispersed phase or in rendering the formulation biocompatible.

Acceptable additives include acidifying agents, alkalizing agents, antimicrobial preservatives, antioxidants, buffering agents, chelating agents, suspending and/or viscosity-increasing agents, including triodobenzene derivative, such as iohexol or iopamidol, tonicity agents, acacia, agar, alginic acid, aluminum mono-stearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xanthum gum.

In some embodiments, the oxygen transport substance may be an emulsion of about 0.1% to about 8% w/v dodecafluoropentane. In other embodiments, the oxygen transport substance may be an emulsion of about 0.1% to about 1.5% w/v dodecafluoropentane. In yet other embodiments, the oxygen transport substance may be an emulsion of about 0.5% to about 2.5% w/v dodecafluoropentane. In additional embodiments, the oxygen transport substance may be an emulsion of about 1% to about 3% w/v dodecafluoropentane. In preferred embodiments, the oxygen transport substance may be an emulsion of about 1% to about 5% w/v dodecafluoropentane.

The emulsions may be formed by comminuting a suspension of the dispersed phase in the continuous phase by the application of mechanical, manual, or acoustic energy. Comminuting comprises the process of forming a colloidal dispersion by mixing the liquid dispersed and continuous phases together and then causing a decrease in size of the particles of the dispersed phase from large particles to the size required, using mechanical energy generated by mixing manually, mechanically, or by the action of ultrasound. Appropriate mixing can be achieved in a Microfluidic's Model 1 10 Microfluidizer apparatus, as described in U.S. Pat. No. 4,533,254, incorporated herein by reference.

Depending on the particular compound, the microbubbles are stabilized to last in the bloodstream for a time ranging from a few minutes to several hours. It will be appreciated by those of skill in the art that the size of the microbubbles formed can be controlled by the manufacturing process to be sufficiently small so as not to obstruct the systemic or pulmonary capillaries and to pass through or around vessels occluded to flow of larger red blood cells. In an exemplary embodiment, the oxygen transport substance may be microbubbles comprising DDFPe, formulated as an emulsion of 250 nanometer droplets.

An oxygen transport substance of the disclosure may be administered to a subject by parenteral administration such as via intravenous injection, intra-arterial, intramuscular, intraperitoneal, intraventricular, epidural, intracranial injection, and infusion techniques. In one embodiment, the oxygen transport substance may be administered to a subject by intra-arterial injection. In another embodiment, the oxygen transport substance may be administered to a subject by intramuscular injection. In still another embodiment, the oxygen transport substance may be administered to a subject via intraperitoneal injection. In another embodiment, the oxygen transport substance may be administered to a subject by intraventricular injection. In yet another embodiment, the oxygen transport substance may be administered to a subject by intracranial injection. In another embodiment, the oxygen transport substance may be administered to a subject by epidural injection. In preferred embodiments, the oxygen transport substance may be administered to a subject intravenously.

In some embodiments, the oxygen transport substance may be administered in a bolus. In other embodiments, the oxygen transport substance may be administered continuously. In yet other embodiments, the oxygen transport substance may be administered in a combination of a bolus and continuously. Non-limiting examples of continuous administration may include infusion.

The oxygen transport substance may be administered to a subject once, or multiple times. In some preferred embodiments, the oxygen transport substance may be administered once. In other preferred embodiments, the oxygen transport substance may be administered multiple times. For instance, the oxygen transport substance may be administered 2, 3, 4, 5, 6, 7, 8, 9, 10, 20 or more times. In some embodiments, the oxygen transport substance may be administered 2, 3, 4, 5, 6, 7, 8, 9, or 10 times. In other embodiments, the oxygen transport substance may be administered 9, 10, 11, 12, 13, 14, 15, 16, 17, 18, 19, 20 or more times. In preferred embodiments, the oxygen transport substance may be administered 2, 3, 4, 5, or 6 times.

Yet another preferred method of administration is by sustained IV infusion. When administered by IV infusion an initial bolus or slow IV push loading dose may be administered generally ranging from about 0.01 to about 0.6 cc per kg body weight with 2% w/vol DDFPe. More preferably the loading dose is from about 0.05 to about 0.3 cc per kg. Thereafter the material is infused IV for between about 1 hour and up to 24 hours and even longer depending upon the subject's condition. For sustained infusion the material is generally infused at rates from about 0.01 to about 0.3 cc per kg and more preferably from about 0.025 to about 0.1 cc per kg per hour.

When administered multiple times, the oxygen transport substance may be administered at regular intervals or at intervals that may vary during the treatment of a subject. In some embodiments, the oxygen transport substance may be administered multiple times at intervals that may vary during the treatment of a subject. In preferred embodiments, the oxygen transport substance may be administered multiple times at regular intervals. In some alternatives of the preferred embodiments, the oxygen transport substance may be administered at intervals of about 10, 20, 30, 40, 50, 60, 70, 80, 90, 100, 110, 120, 130, 140, 150, 160, 170, 180, or more minutes. In other alternatives of the preferred embodiments, the oxygen transport substance may be administered at intervals of about 10, 20, 30, 40, 50, 60, 70, 80, 90, 100 or more minutes. In yet other alternatives of the preferred embodiments, the oxygen transport substance may be administered at intervals of about 80, 90, 100 or more minutes. In other alternatives of the preferred embodiments, the oxygen transport substance may be administered at intervals of about 80, 81, 82, 83, 84, 85, 86, 87, 88, 89, 90, 91, 92, 93, 94, 95, 96, 97, 98, 99, 100 or more minutes. In exemplary embodiments, the oxygen transport substance may be administered at intervals of about 90 minutes.

The oxygen transport substance may be administered to a subject undergoing ischemia, prior to development of ischemia, or administered prior to development of ischemia and continued throughout an ischemic episode. For instance, administration of the oxygen transport substance to a subject may be administered prior to development of ischemia when the subject is undergoing a medical procedure that increases the risk of ischemia due to vessel occlusion, or when the subject is at high risk for developing an occluded blood vessel as described in Section 1(b) above. In some embodiments, the oxygen transport substance may be administered to a subject undergoing ischemia. In other embodiments, the oxygen transport substance may be administered to a subject prior to development of ischemia. In yet other embodiments, the oxygen transport substance may be administered to the subject prior to development of ischemia and continued throughout an ischemic episode. In preferred embodiments, the oxygen transport substance may be administered to a subject before a medical procedure that increases the risk of vessel occlusion is performed. In other preferred embodiments, the oxygen transport substance may be administered to a subject at high risk for developing an occluded blood vessel prior to onset of symptoms of an occluded blood vessel.

In some embodiments, the oxygen transport substance may be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 minutes or more prior to development of ischemia. In one embodiment, the oxygen transport substance may be administered about 1, 2, 3, 4, 5, 6, 7, 8, 9, 10, 11, 12, 13, 14, 15, 16, 17, 18, 19, 20 minutes prior to development of ischemia. In another embodiment, the oxygen transport substance may be administered about 15, 16, 17, 18, 19, 20, 21, 22, 23, 24, 25, 26, 27, 28, 29, 30 minutes prior to development of ischemia. In yet another embodiment, the oxygen transport substance may be administered about 25, 26, 27, 28, 29, 30, 31, 32, 33, 34, 35, 36, 37, 38, 39, 40 minutes prior to development of ischemia. In another embodiment, the oxygen transport substance may be administered about 35, 36, 37, 38, 39, 40, 41, 42, 43, 44, 45, 46, 47, 48, 49, 50 minutes prior to development of ischemia. In an additional embodiment, the oxygen transport substance may be administered about 45, 46, 47, 48, 49, 50, 51, 52, 53, 54, 55, 56, 57, 58, 59, 60 minutes prior to development of ischemia. In yet another embodiment, the oxygen transport substance may be administered about 55, 56, 57, 58, 59, 60, 61, 62, 63, 64, 65, 66, 67, 68, 69, 70 minutes prior to development of ischemia. In another embodiment, the oxygen transport substance may be administered about 65, 66, 67, 68, 69, 70, 71, 72, 73, 74, 75, 76, 77, 78, 79, 80 minutes prior to development of ischemia. In yet another embodiment, the oxygen transport substance may be administered about 75, 76, 77, 78, 79, 80, 81, 82, 83, 84, 85, 86, 87, 88, 89, 90 minutes prior to development of ischemia. In an additional embodiment, the oxygen transport substance may be administered about 85, 86, 87, 88, 89, 90, 91, 92, 93, 94, 95, 96, 97, 98, 99, 100 minutes prior to development of ischemia. In still another embodiment, the oxygen transport substance may be administered about 95, 96, 97, 98, 99, 100, 101, 102, 103, 104, 105, 106, 107, 108, 109, 110 minutes or more prior to development of ischemia. In a preferred embodiment, the oxygen transport substance may be administered about 25, 26, 27, 28, 29, 30, 31, 32, 33, 34, 35 minutes prior to development of ischemia.

In some embodiments, the oxygen transport substance may be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 minutes, or 1, 2, 3, 4, 5, or 6 hours or more after the onset of ischemia. In one embodiment, the oxygen transport substance may be administered about 1, 2, 3, 4, 5, 6, 7, 8, 9, 10, 11, 12, 13, 14, 15, 16, 17, 18, 19, 20 minutes after the onset of ischemia. In another embodiment, the oxygen transport substance may be administered about 15, 16, 17, 18, 19, 20, 21, 22, 23, 24, 25, 26, 27, 28, 29, 30 minutes after the onset of ischemia. In yet another embodiment, the oxygen transport substance may be administered about 25, 26, 27, 28, 29, 30, 31, 32, 33, 34, 35, 36, 37, 38, 39, 40 minutes after the onset of ischemia. In another embodiment, the oxygen transport substance may be administered about 35, 36, 37, 38, 39, 40, 41, 42, 43, 44, 45, 46, 47, 48, 49, 50 minutes after the onset of ischemia. In yet another embodiment, the oxygen transport substance may be administered about 1, 2, 3, 4, 5, or 6 hours or more after the onset of ischemia. In a preferred embodiment, the oxygen transport substance may be administered about 45, 46, 47, 48, 49, 50, 51, 52, 53, 54, 55, 56, 57, 58, 59, 60, 61, 62, 63, 64, or 65 minutes after the onset of ischemia. In another preferred embodiment, the oxygen transport substance may be administered about 1, 2, 3, 4, 5, or 6 hours or more after the onset of ischemia. In an exemplary embodiment, the oxygen transport substance may be administered less than about 1 hour after the onset of ischemia. In another exemplary embodiment, the oxygen transport substance may be administered about 1, 2, or 3 hours after the onset of ischemia.

In some embodiments, the oxygen transport substance may be administered to the subject in an amount of about 0.1, 0.2, 0.3, 0.4, 0.5, 0.6, 0.7, 0.8, 0.9, 1, or about 1.1 mL per kilogram of the subject. In other embodiments, the oxygen transport substance may be administered to the subject in an amount of about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or about 0.2 mL per kilogram of the subject. In yet other embodiments, the oxygen transport substance may be administered to the subject in an amount of about 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, or about 0.3 mL per kilogram of the subject. In still other embodiments, the oxygen transport substance may be administered to the subject in an amount of about 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, or about 0.4 mL per kilogram of the subject. In other embodiments, the oxygen transport substance may be administered to the subject in an amount of about 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or about 0.5 mL per kilogram of the subject. In yet other embodiments, the oxygen transport substance may be administered to the subject in an amount of about 0.41, 0.42, 0.43, 0.44, 0.45, 0.46, 0.47, 0.48, 0.49, 0.5, 0.51, 0.42, 0.53, 0.54, 0.55, 0.56, 0.57, 0.58, 0.59, or about 0.6 mL per kilogram of the subject. In still other embodiments, the oxygen transport substance may be administered to the subject in an amount of about 0.001, 0.005, 0.01, 0.015, 0.02, 0.025, 0.03, 0.035, 0.04, 0.045, 0.05, 0.055, 0.06, 0.065, 0.07, 0.075, 0.08, 0.085, 0.09, or about 0.1 mL per kilogram of the subject. In some preferred embodiments, the oxygen transport substance may be administered to the subject in an amount of about 0.6 mL per kilogram of the subject. In other preferred embodiments, the oxygen transport substance may be administered to the subject in an amount of about 0.3 mL per kilogram of the subject. In yet other preferred embodiments, the oxygen transport substance may be administered to the subject in an amount of about 0.1 mL per kilogram of the subject.

The oxygen transport substance of the invention may be administered in combination with other treatments for ischemia or treatments that may increase oxygenation of tissue. Non-limiting examples of treatments for ischemia or treatments that may increase oxygenation of tissue may include oxygen inhalation, administration of blood, thrombolytics or anticoagulants, and reducing the temperature of the tissue.

Generally speaking, an administration of an oxygen transport substance of the invention may be used to reduce infarct volume while a secondary treatment is used to resolve the occlusion. Importantly, a composition of the invention may be used to reduce infarct volume during ischemia even though the occlusion is not resolved. Hence, it is envisioned that a composition of the invention is administered first, to protect tissue, and then treatments to resolve the occlusion may be administered.

In some embodiments, the oxygen transport substance of the invention may be administered in combination with blood. In other embodiments, the oxygen transport substance of the invention may be administered in combination with oxygen inhalation. In yet other embodiments, the oxygen transport substance of the invention may be administered in combination with one or more anticoagulant. Non-limiting examples of anticoagulants may include vitamin K antagonists such as acenocoumarol, coumatetralyl, dicoumarol, ethyl biscoumacetate, phenprocoumon, warfarin, dorindione, diphenadione, phenindione, antiplatelet compounds such as abciximab, eptifibatide, tirofiban, clopidogrel, prasugrel, ticlopidine, cangrelor, elinogrel, ticagrelor, beraprost, prostacyclin, iloprost, treprostinil, acetylsalicylic acid (aspirin), aloxiprin, carbasalate calcium, indobufen, triflusal, dipyridamole, picotamide, terutroban, cilostazol, dipyridamole, triflusal, cloricromen, ditazole, inhibitors of factor Xa such as bemiparin, certoparin, dalteparin, enoxaparin, nadroparin, parnaparin, reviparin, tinzaparin, fondaparinux, idraparinux, danaparoid, sulodexide, dermatan sulfate, apixaban, betrixaban, edoxaban, otamixaban, rivaroxaban, peviparin, YM466, direct thrombin II inhibitors such as bivalirudin, lepirudin, desirudin, argatroban, dabigatran, melagatran, ximelagatran, REG1, defibrotide, ramatroban, antithrombin III, and protein C (Drotrecogin alfa), and thrombolytic drugs such as plasminogen activators (tPA; alteplase, reteplase, tenecteplase), antistreptase, Urokinase, Saruplase, streptokinase, anistreplase, monteplase, ancrod, fibrinolysin, brinase, aspirin and salicylate.

In other embodiments, the oxygen transport substance of the invention may be administered in combination with one or more thrombolytic. Non-limiting examples of thrombolytics may include plasminogen activators (tPA; alteplase, reteplase, tenecteplase), antistreptase, Urokinase, Saruplase, streptokinase, anistreplase, monteplase, ancrod, fibrinolysin, and brinase.

In some preferred embodiments, the oxygen transport substance of the invention may be administered in combination with an anticoagulant or thrombolytic selected from the group consisting of tissue plasminogen activators, antistreptase, streptokinase, urokinase, and combinations thereof. In one alternative of the preferred embodiments, the oxygen transport substance of the invention may be administered in combination with tPA. In exemplary embodiments, tPA may be administered after administration of the oxygen transport substance of the invention, followed by a second dose of the oxygen transport substance as described in the examples.

In other embodiments, a composition of the invention may be combined with lowering the temperature of the tissue suffering the ischemic event. In all instances, however, the tissue temperature is lowered to no less than 29° C. For instance, the tissue temperature may be lowered to about 30, 31, 32, 33, 34, 35, or 36° C.

In some aspects, the present disclosure provides a combination comprising a dodecafluoropentane emulsion and a thrombolytic. The dodecafluoropentane emulsion and the thrombolytic are as described hereinabove. In some embodiments, the combination comprises a thrombolytic selected from the group consisting of tissue plasminogen activators, antistreptase, streptokinase, urokinase, and combinations thereof. In preferred embodiments, the combination comprises a dodecafluoropentane emulsion and tPA. Generally speaking, the effective amount of tPA may be determined using methods commonly known in the art.

A cardioprotectant may be included in the emulsion or co-administered with the emulsion. Useful cardioprotectants include sodium nitrite, nitric oxide, and nitric oxide donors such as the NO donor S-nitroso-N-acetylpenicillamine (SNAP). Agonists that bind to Gi or Gq protein coupled receptors may be used in the invention. They include bradykinin, opioids, and adenosine. Acetylcholine (ACh) may be utilized as a cardioprotectant, (2-acetoxybenzoate 2-[1-nitroxy-methyl]-phenyl ester, and sildenafil. Although statins are generally though of as lipid-lowering agents, statins also upregulate NOS activity predominantly by posttranscriptional mechanisms and therefore may be used to stimulate nitric oxide release. Useful statins include but are not limited to, simvastatin and fluvastatin. Natriuretic peptides and tricarbonylchloro-(glycinato) ruthenium II (CORM-3) may also be used as cardioprotectants. Beta blockers may be employed as cardioprotectants and include but are not limited to metoprolol tartrate, Lopressor. Ionotropic agents may be employed in the invention including but not limited to inamirinone lactate (Incor). Additional cardioprotecant agents include Thymosin B4, dexrazoxane, vitamin E, selenium (e.g. selenite) and glutathione. Erythropoietin, e.g. epoetin alfa may also be used as a cardioprotecant with DDFPe or after administration of DDFPe.

The following examples are presented to further illustrate to persons skilled in the art how to make and use the invention. These examples are not intended as a limitation, however, upon the scope of the invention.

### Example I

A fifty-five year old male has crushing chest pain. An Advanced Life Support unit arrives at the patient's home and he is taken by ambulance to the hospital. Intravenous access is obtained, supplemental oxygen is provided, pulse oximetry is obtained and the patients is administered aspirin en route. He is given nitroglycerin for active chest pain sublingually. Telemetry prehospital ECG, is obtained and interpreted by staff at the hospital. Acute ST elevation MI is diagnosed (STEMI). The ambulance is equipped with 2% weight/volume dodecafluoropentane emulsion (DDFPe). An amount of 0.1 cc per kg of DDFPe is administered as slow IV push. Chest pain subsides and critical oxygen is delivered to at risk myocardium, decreasing size of the myocardial infarction and preserving more myocardial tissue.

### Example 2

A sixty-eight year woman residing in a rural area has crushing chest pain. The ambulance arrives and STEMI is diagnosed via telemetry. She is administered aspirin and nitroglycerin. It is an approximate 60 minute journey by ambulance to arrive at the closest hospital offering percutaneous coronary intervention (PCI). In addition to the aspirin she is administered tissue-type plasminogen activator (t-PA) and heparin as well as 0.1 cc/kg of DDFPe IV. After arrival at the PCI hospital she is stabilized and treated with angioplasty and stent.

While the preferred embodiments of the present invention have been illustrated in detail, it should be apparent that modifications and adaptations to those embodiments may occur to one skilled in the art without departing from the scope of the present invention as set forth herein.

1. A composition for treating a subject who has incurred or is incurring damage to the heart, comprising:
a perfluorocarbon; and
a lipid.

2. The composition of claim 1, wherein said perfluorocarbon consists essentially of an emulsion of dodecafluoropentane in water.

3. The composition of claim 2, wherein said dodecafluoropentane emulsion comprises a 2 percent weight/volume dodecafluoropentane emulsion in water.

4. The composition of claim 2, wherein said dodecafluoropentane emulsion further comprises a surfactant.

5. The composition of claim 4, wherein said surfactant comprises a phospholipid.

6. The composition of claim 5, wherein said phospholipid comprises a fluorinated phospholipid.

7. The composition of claim 5, wherein said phospholipid comprises an ether lipid.

8. The composition of claim 5, wherein said phospholipid comprises a PEG moiety.

9. (canceled)

10. (canceled)

11. A method for treating a subject who has incurred or is incurring damage to the heart, comprising administering to said subject a therapeutically effective amount of dodecafluoropentane.

12. The method of claim 11, wherein said dodecafluoropentane is administered as a dodecafluoropentane emulsion.

13. (canceled)

14. The method of claim 11, wherein said damage is myocardial damage.

15. The method of claim 11, wherein said damage arises from ischaemia, ischaemia/reperfusion injury, hypoxia, increased cardiac workload or cardiac stress, increased pressure on the heart, a cardiotoxic substance, infection, or a maladaptive response of the heart to injury or disease.

16. (canceled)

17. (canceled)

18. The method of claim 11, wherein administration of therapeutically effective amount of dodecafluoropentane is performed during or after the acute coronary syndrome.

19. The method of claim 18, wherein administration of therapeutically effective amount of dodecafluoropentane is performed immediately after the acute coronary syndrome.

20. The method of claim 18, wherein the acute coronary syndrome is myocardial infarction.

21. The method of claim 11, wherein said method reduces or ameliorates the heart damage, or protects the heart from damage during or from the acute coronary syndrome or ischaemic event.

22. (canceled)

23. (canceled)

24. The method of claim 11, wherein the method is used (i) to prevent or delay the onset or development of heart failure after myocardial infarction (MI); or (ii) to prevent or reduce the extent of MI; or (iii) before, during or after percutaneous coronary intervention (PCI).

25. The method of claim 24, wherein the method is for administration after an acute coronary syndrome but before, during or after restoration of coronary blood flow, preferably immediately before restoration of coronary blood flow or immediately after reopening of thrombotic blood vessels, or during PCI.

26. The method of claim 11, for use in the treatment of acute or chronic heart failure.

27. (canceled)

28. The method of claim 12, wherein said dodecafluoropentane emulsion comprises a 2 percent weight/volume dodecafluoropentane emulsion in water.

29-34. (canceled)
